(19) United States
(12) Patent Application Publication
(10) Pub. No.: US 2026/0115121 A1
(43) Pub. Date: Apr. 30, 2026

(54) COMPOSITION FOR LOCAL ADIPOSE TISSUE ENLARGEMENT AND APPLICATION

(71) Applicant: CHUNTIAN WANG, ANSHAN (CN)

(72) Inventor: CHUNTIAN WANG, ANSHAN (CN)

(21) Appl. No.: 18/928,439

(22) Filed: Oct. 28, 2024

(51) Int. Cl. A61K 8/67; A61K 8/92

(52) U.S. Cl. CPC A61K 8/673; A61K 8/92

(57) ABSTRACT

A composition for local adipose tissue enlargement and application is disclosed, which relates to the technical field of beauty and body care products, comprising 4-10 parts of Anemarrhena asphodeloides Bunge extract, 1-3 parts of vitamin B₁₂, 1-2 parts of geranium essential oil and 1-3 parts of ceramide. Pure plant extract components are selected in the composition, supplemented with vitamin B and ceramide. The effective components can be absorbed by the skin of the breast and the buttocks by external application, rubbing and massage, so as to promote the accumulation of adipose cells, achieve the effect of breast augmentation and buttock augmentation, avoid side effects caused by surgery and injection, and ensure safety and no irritation. Meanwhile, due to the presence of the ceramide, the composition can also maintain the function and the morphology of fibroblasts, maintain or improve the differentiation ability, promote tissue regeneration, and facilitate wound healing.

### TECHNICAL FIELD

The present invention relates to the technical field of beauty and body care products, in particular to a composition for local adipose tissue enlargement and application.

### BACKGROUND

The breast is one of the important reproductive accessories of women. The breast is composed of skin, glands, connective tissue and adipose tissue. The fat of the mature breast accounts for ⅔, the glands account for ⅓, and the amount of the fat determines the size of the breast. The breast can be fuller through stimulation of proliferation of breast adipose cells and gland development.

The first is breast augmentation prosthesis which is a body shaping mode that has the longest period of operation and the most methods in the world. Breast augmentation prosthesis means that an incision is made around the breast, then a sculpted breast prosthesis is inserted, and finally the breast is stitched to achieve the effect. The breast prosthesis is made of silicone gel. However, breast augmentation prosthesis has many major disadvantages. Due to the rejection reaction of a human body for foreign matter, a layer of capsule is formed around the prosthesis, which has no influence in normal circumstances. If the rejection reaction is too serious, capsular contracture and hardening and deformity of the breast may be caused, so there is a great risk of prosthesis displacement or capsular contracture. The second is injection of collagen or hyaluronic acid, but collagen can also produce rejection reaction. The injection of hyaluronic acid in the breast will become a diagnostic obstacle in the diagnosis of breast cancer. The injected hyaluronic acid is mostly diagnosed as many black cysts (pustules). This is a phenomenon of subcutaneous tissue caused by repeated injection of hyaluronic acid. In hyaluronic acid injection, the subcutaneous tissue of the hyaluronic acid becomes a problem. The residual hyaluronic acid injected into the subcutaneous tissue and not absorbed by the body is perceived as foreign matter. Many patients have undergone surgery to remove the breast and the tissue around the breast including the injected hyaluronic acid. It is believed that it will become a social problem in the future. The same is true for buttock augmentation.

Therefore, there is an urgent need to develop an external composition that can achieve the effect of promoting local adipose accumulation without injection or breast augmentation prosthesis and buttock augmentation.

### SUMMARY

In view of this, the present invention is proposed.

In order to realize the above purpose, the present invention adopts the following technical solution:

A first aspect of an embodiment of the present invention provides a composition for local adipose tissue enlargement, comprising:

| | | |
|---|---|---|
| Anemarrhena asphodeloides Bunge extract | 4-10 | parts |
| vitamin B₁₂ | 1-3 | parts |
| geranium essential oil | 1-2 | parts |
| ceramide | 1-3 | parts. |

In a preferred embodiment, the composition comprises the following components by optimal weight parts:

| | |
|---|---|
| Anemarrhena asphodeloides Bunge extract | 5 parts |
| vitamin B₁₂ | 1 part |
| geranium essential oil | 1 part |
| ceramide | 2 parts. |

A second aspect of the embodiment of the present invention provides an application of the composition for local adipose tissue enlargement in preparation of creams for breast augmentation and buttock augmentation.

A third aspect of the embodiment of the present invention provides an application of the composition for local adipose tissue enlargement in preparation of creams for maintaining the function of fibroblasts and promoting wound repair.

A fourth aspect of the embodiment of the present invention provides a cream, comprising 85-95 parts of cream matrix, 4-10 parts of Anemarrhena asphodeloides Bunge extract, 1-3 parts of vitamin B₁₂, 1-2 parts of geranium essential oil and 1-3 parts of ceramide.

A fifth aspect of the embodiment of the present invention provides a preparation method of the cream, comprising a process:
1) preparing the Anemarrhena asphodeloides Bunge extract for later use;
2) weighing the cream matrix, the Anemarrhena asphodeloides Bunge extract, the vitamin B₁₂, the geranium essential oil and the ceramide according to weight parts, mixing and stirring into a uniform system to obtain the cream.

In a preferred embodiment, a preparation process of the Anemarrhena asphodeloides Bunge extract is as follows: taking Anemarrhena asphodeloides Bung leaves, washing and drying; then boiling Anemarrhena asphodeloides Bung in 6-8 times of water for 2 h and filtering; boiling filter residues in 6-8 times of water for 2 h and filtering; combining the filtrate; and concentrating under reduced pressure for 1 h to obtain the Anemarrhena asphodeloides Bung extract.

In a preferred embodiment, the cream matrix, the geranium essential oil, the vitamin B₁₂ and the ceramide are all commercially available.

According to the above technical solution, compared with the prior art, the present invention discloses and provides a composition for local adipose tissue enlargement. Pure plant extract components are selected in the composition, supplemented with vitamin B and ceramide. The effective components can be absorbed by the skin of the breast and the buttocks by external application, rubbing and massage, so as to promote the accumulation of adipose cells, achieve the effect of breast augmentation and buttock augmentation, avoid side effects caused by surgery and injection, and ensure safety and no irritation. At the same time, due to the presence of the ceramide, the composition can also maintain the function and the morphology of fibroblasts, maintain or improve the differentiation ability, promote tissue regeneration, and facilitate wound healing.

### DETAILED DESCRIPTION

The technical solutions in the present invention will be clearly and fully described below in combination with the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

The effects of the component in the embodiment of the present invention are as follows:

The Anemarrhena asphodeloides Bung extract contains a large amount of sarsasapogenin which can improve the activity of superoxide dismutase, and has the effects of promoting glucose oxidation, lowering blood sugar, and resisting platelet aggregation and pathogenic microorganisms. However, the present invention recently has found that the Anemarrhena asphodeloides Bung extract can promote the accumulation of local adipose tissue.

The vitamin B₁₂ promotes the development and maturity of red blood cells, so that the somatic hematopoietic function is in a normal state to prevent pernicious anemia. The vitamin B₁₂ maintains nervous system health, and can increase the utilization rate of folic acid and promote the metabolism of carbohydrates, fats and proteins. The vitamin B₁₂ has the effect of activating amino acid, promotes the biosynthesis of nucleic acid, can promote the synthesis of proteins, and plays an important role in the growth and development of infants and young children. The vitamin B₁₂ metabolizes fatty acid so that fat, carbohydrates and protein are properly used by the body.

The geranium essential oil promotes blood circulation, dredges the mammary gland, and promotes adipose accumulation.

The ceramide is a substance that exists in the skin, and is a major part that forms an intercellular matrix. The ceramide forms a reticular structure in the stratum corneum, retains moisture in the skin and keeps the skin hydrated.

The cream matrix, the geranium essential oil, the vitamin B₁₂ and the ceramide are all commercially available.

### Embodiment 1

A cream for breast augmentation and buttock augmentation comprises a composition for local adipose tissue enlargement and a cream matrix, comprising:

| | | |
|---|---|---|
| cream matrix | 85 | parts |
| Anemarrhena asphodeloides Bunge extract | 10 | parts |
| vitamin B₁₂ | 2 | parts |
| geranium essential oil | 1 | part |
| ceramide | 2 | parts. |

### Embodiment 2

A cream for breast augmentation and buttock augmentation comprises a composition for local adipose tissue enlargement and a cream matrix, comprising:

| | | |
|---|---|---|
| cream matrix | 91 | parts |
| Anemarrhena asphodeloides Bunge extract | 5 | parts |
| vitamin B₁₂ | 1 | part |
| geranium essential oil | 1 | part |
| ceramide | 2 | parts. |

### Embodiment 3

A cream for breast augmentation and buttock augmentation comprises a composition for local adipose tissue enlargement and a cream matrix, comprising:

| | | |
|---|---|---|
| cream matrix | 95 | parts |
| Anemarrhena asphodeloides Bunge extract | 8 | parts |
| vitamin B₁₂ | 3 | parts |
| geranium essential oil | 2 | parts |
| ceramide | 1 | part. |

### Embodiment 4

A cream for breast augmentation and buttock augmentation comprises a composition for local adipose tissue enlargement and a cream matrix, comprising:

| | | |
|---|---|---|
| cream matrix | 87 | parts |
| Anemarrhena asphodeloides Bunge extract | 7 | parts |
| vitamin B₁₂ | 3 | parts |
| geranium essential oil | 3 | parts |
| ceramide | 1 | part. |

The preparation process of the above cream is:
1) Anemarrhena asphodeloides Bung leaves were taken, washed and dried; then Anemarrhena asphodeloides Bung was boiled in 6-8 times of water for 2 h and filtered; filter residues were boiled in 6-8 times of water for 2 h and filtered; and the filtrate was combined and concentrated under reduced pressure for 1 h to obtain the Anemarrhena asphodeloides Bung extract for later use;
2) the cream matrix, the Anemarrhena asphodeloides Bunge extract, the vitamin B₁₂, the geranium essential oil and the ceramide were weighed according to weight parts, mixed and stirred into a uniform system to obtain the cream.

### Embodiment 5 Skin Irritation Experiment of Cream

30 female volunteers aged between 18 and 35 were selected, wherein 19 volunteers have sensitive skin and 11 volunteers have non-sensitive skin, but the volunteers with sensitive and non-sensitive skin have oily skin, normal skin and dry skin. The creams of embodiments 1 to 4 were applied to the 30 volunteers. The left hand backs were applied and massaged until the creams were completely absorbed. The creams were used once a day for consecutive 30 days. The number of persons with allergic symptoms is shown in Table 1.

TABLE 1
Number of Persons with Allergic Symptoms

| | Sensitive Skin | | | | Non-Sensitive Skin | | | |
|---|---|---|---|---|---|---|---|---|
| | | Swelling | | | | Swelling | | |
| | | and | | | | and | | |
| | Red | itching | Pricking | Erythema | Red | itching | Pricking | Erythema |
| Embodiment 1 | 0 | 0 | 0 | 0 | 0 | 0 | 0 | 0 |
| Embodiment 2 | 0 | 0 | 0 | 0 | 0 | 0 | 0 | 0 |
| Embodiment 3 | 0 | 0 | 0 | 0 | 0 | 0 | 0 | 0 |
| Embodiment 4 | 0 | 0 | 0 | 0 | 0 | 0 | 0 | 0 |

It can be seen from Table 1 that the cream of the present invention is safe, gentle, and non-irritating to skin, and can be used safely.

### Embodiment 6 Verification of Breast Augmentation and Buttock Augmentation Effects of Cream

Embodiments 1-4 are taken as experimental groups 1-4 respectively. At the same time, reference examples 1-3 are set:
Reference example 1: Compared with experimental group 2, the Anemarrhena asphodeloides Bunge extract was removed, the weight parts of the geranium essential oil were changed to 6 parts, and other components were unchanged;
Reference example 2: Compared with experimental group 2, the geranium essential oil was removed, the weight parts of the Anemarrhena asphodeloides Bunge extract were changed to 6 parts, and other components were unchanged;
Reference example 3: Compared with experimental group 2, the Anemarrhena asphodeloides Bunge extract was replaced by Anemarrhena asphodeloides root extract, and the ratio of the components was unchanged.

70 women aged 18-40 were selected. According to statistics, 70 women were not satisfied with their breast sizes and buttock fullness to different degrees. Therefore, through notification of the specific information, by following the voluntary principle, 70 women were divided into 7 groups with 10 women in each group to try the cream products of the experimental groups 1-4 and the reference examples 1-3 respectively. The enlargement of the breast and the buttock was observed after continuous use for 28 d and 56 d, as shown in Table 2.

TABLE 2

| | |
|---|---|
| 28 d (mm³) | 56 d (mm³) |

| | Breast | Buttock | Breast | Buttock |
|---|---|---|---|---|
| Experimental | 92.34 | 86.12 | 220.31 | 231.84 |
| group 1 | | | | |
| Experimental | 96.17 | 88.57 | 229.47 | 235.79 |
| group 2 | | | | |
| Experimental | 85.73 | 84.57 | 217.16 | 219.15 |
| group 3 | | | | |
| Experimental | 81.62 | 81.63 | 209.73 | 224.94 |
| group 4 | | | | |
| Reference | 64.79 | 46.18 | 149.30 | 147.65 |
| example 1 | | | | |
| Reference | 59.92 | 52.64 | 135.86 | 154.83 |
| example 2 | | | | |
| Reference | 75.41 | 60.71 | 139.91 | 169.27 |
| example 3 | | | | |

It can be seen from Table 2 that the cream of the present invention can promote local adipose accumulation, and then achieve significant breast augmentation and buttock augmentation effects. Moreover, according to the comparison of the experimental groups 1-4 and the reference examples 1-2, the Anemarrhena asphodeloides Bunge extract and the geranium essential oil in the present invention have the synergistic effect which is unexpected.

### Embodiment 7 Wound Healing Experiment

An SD rat was selected, and anesthetized with isoflurane. The hair on the back of the rat was shaved with a shaver. The back was moulted by a hair removal cream. Then, an experimental region on the back of the rat was cleaned and disinfected with iodophor. Later, a skin sampler with a diameter of 10 mm was rotatably pressed on the back of the rat (to basically ensure the same position of each model) to form a small notch. The skin was removed by eye tweezers and eye scissors to form a circular wound with a diameter of 10 mm. Measurement of wound area: the rat was placed flat and photographed at the same height by using an industrial high-definition camera, and the initial wound measurement was performed. Subsequently, the products of embodiments 1-4 and the reference examples 1-2 were used. The rat was observed and recorded every 2 days. The result was calculated according to the wound healing rate=(initial wound area−wound area on the day of measurement)/(initial wound area)*100%. The measured results are shown in Table 3.

Embodiments 1-4 are taken as experimental groups 1-4 respectively. At the same time, reference examples 1-2 are set:
Reference example 1: Compared with experimental group 2, the ceramide was removed, the vitamin B₁₂ was changed to 3 parts, and other components were unchanged;
Reference example 2: Compared with experimental group 2, the vitamin B₁₂ was removed, the weight parts of the ceramide were changed to 6 parts, and other components were unchanged.

TABLE 3
Wound Healing Rate (%)

| | 2 d | 4 d | 6 d | 8 d |
|---|---|---|---|---|
| Experimental | 40.25 | 61.79 | 83.86 | 99.23 |
| group 1 | | | | |
| Experimental | 42.13 | 65.17 | 85.49 | 100 |
| group 2 | | | | |
| Experimental | 40.68 | 62.57 | 82.44 | 98.34 |
| group 3 | | | | |
| Experimental | 41.18 | 63.38 | 84.65 | 99.16 |
| group 4 | | | | |
| Reference | 21.69 | 36.75 | 50.79 | 65.14 |
| example 1 | | | | |
| Reference | 24.57 | 34.97 | 46.72 | 60.64 |
| example 2 | | | | |

It can be seen from Table 3 that the cream of the present invention can promote rapid wound healing.

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A composition for local adipose tissue enlargement, comprising:
Anemarrhena asphodeloides Bunge extract 4-10 parts

| | |
|---|---|
| vitamin B₁₂ | 1-3 parts |
| geranium essential oil | 1-2 parts |
| ceramide | 1-3 parts. |

2. The composition for local adipose tissue enlargement according to claim 1, comprising the following components by optimal weight parts:
Anemarrhena asphodeloides Bunge extract 5 parts

| | |
|---|---|
| vitamin B₁₂ | 1 part |
| geranium essential oil | 1 part |
| ceramide | 2 parts. |

3. An application of the composition for local adipose tissue enlargement according to claim 1 in preparation of creams for breast augmentation and buttock augmentation.

4. An application of the composition for local adipose tissue enlargement according to claim 1 in preparation of creams for maintaining the function of fibroblasts and promoting wound repair.

5. A cream, comprising 85-95 parts of cream matrix, 4-10 parts of Anemarrhena asphodeloides Bunge extract, 1-3 parts of vitamin B₁₂, 1-2 parts of geranium essential oil and 1-3 parts of ceramide.

6. A preparation method of the cream according to claim 5, comprising a process:
1) preparing the Anemarrhena asphodeloides Bunge extract for later use;
2) weighing the cream matrix, the Anemarrhena asphodeloides Bunge extract, the vitamin B₁₂, the geranium essential oil and the ceramide according to weight parts, mixing and stirring into a uniform system to obtain the cream.

7. The preparation method of the cream according to claim 6, wherein a preparation process of the Anemarrhena asphodeloides Bunge extract is as follows: taking Anemarrhena asphodeloides Bung leaves, washing and drying; then boiling Anemarrhena asphodeloides Bung in 6-8 times of water for 2 h and filtering; boiling filter residues in 6-8 times of water for 2 h and filtering; combining the filtrate; and concentrating under reduced pressure for 1 h to obtain the Anemarrhena asphodeloides Bung extract.

8. The preparation method of the cream according to claim 7, wherein the cream matrix, the geranium essential oil, the vitamin B₁₂ and the ceramide are all commercially available.
